(19) United States
(12) Patent Application Publication
(10) Pub. No.: US 2013/0096421 A1
(43) Pub. Date: Apr. 18, 2013

(54) MARKER IDENTIFICATION DURING GAMMA OR POSITRON IMAGING WITH APPLICATION TO INTERVENTIONAL PROCEDURES

(71) Applicant: Benjamin Lawrence WELCH, Hampton, VA (US)

(72) Inventor: Benjamin Lawrence WELCH, Hampton, VA (US)

(73) Assignee: Dilon Technologies, Inc.

(21) Appl. No.: 13/271,599

(22) Filed: Oct. 12, 2011

(51) Int. Cl. A61B 6/00; G09G 5/02; G09G 5/00

(52) U.S. Cl. 600/424; 345/634; 345/596

(57) ABSTRACT

A method is provided for identifying a marker by co-registering gamma or positron images. The method is capable of being applied during interventional procedures and includes steps for detecting a first two-dimensional image at a first energy level, detecting a second two-dimensional image at a second energy level, displaying simultaneously the first and second two-dimensional images, and selecting display coefficients for the first and second two-dimensional images

### RELATED APPLICATION(S)

This application is related to U.S. patent application Ser. No. 12/218,602, entitled “Gamma Guided Stereotactic Localization System,” which was filed on Jul. 16, 2008 and is incorporated entirely herein by reference.

### FIELD OF INVENTION

The present invention relates generally to the field of imaging of suspected cancer and, in particular, to the field of a gamma or positron imaging method and system used to guide a physician in the removal of tissue samples for biopsy.

### BACKGROUND OF INVENTION

Mammographic imaging is well-established as the primary screening modality for breast cancer. A suspicious finding on a mammographic examination may lead to imaging with another modality to further investigate the suspicious finding and ultimately to a biopsy being performed to confirm that cancer is or is not present. The other modalities may include a diagnostic mammogram, an ultrasound (US) examination, a magnetic resonance imaging (MRI) procedure, or a nuclear medicine procedure (known as scintimammography). Depending on the nature of the finding and the imaging system with which it was found, the surgeon or radiologist may be guided in the removal of tissue for pathological examination by one of these imaging systems. Breast biopsy systems have been produced and marketed which rely on x-ray guidance, US guidance, and MRI guidance.

Mammograms are x-rays that image tissue densities, not cancer activity. It can be difficult to identify cancerous lesions using mammography, especially when patients have dense breast tissue, multiple suspicious lesions or clusters of microcalcifications, palpable lesions not detected by mammography or ultrasound, post-surgical or post-therapeutic mass, implants, or have been taking Hormone Replacement Therapy.

MRI has shown usefulness as a next-step imaging modality for difficult-to-diagnose cases. Much like x-ray mammography, breast MRI relies on anatomical or structural information, but provides much more detailed images. It is limited, however, by its highly variable specificity, which can range from below 37% to 97%. Combined with its high sensitivity, it is expensive, may require multiple days to complete, and produces a high false positive rate.

Ultrasound is also commonly utilized as a next-step after a questionable mammogram and is good at determining if a suspect mass is solid or fluid-filled. However, ultrasound demonstrates a low specificity rate that can produce misleading results and indicate biopsy where one may not be needed.

Although biopsy systems employing x-ray, ultrasound, and MRI modalities exist, there remains a need for achieving further accuracy in determining the location of potentially cancerous lesions and for the accurate guidance of biopsy systems to the cancerous lesions.

### SUMMARY OF THE INVENTION

Accordingly, the present invention relates to a gamma or positron imaging method and system used to guide a physician in the removal of tissue samples for biopsy that substantially obviates one or more problems due to limitations and disadvantages of the related art.

In an exemplary embodiment, provided is a computer-implemented method for co-registering gamma or positron images, including: (a) generating a first two-dimensional image at a first energy level; (b) generating a second two-dimensional image at a second energy level; (c) displaying simultaneously the first and second two-dimensional images; and (d) selecting display coefficients for the first and second two-dimensional images.

In yet another exemplary embodiment, the first two-dimensional image corresponds to the location of a needle.

In yet another exemplary embodiment, the second two-dimensional image corresponds to the location of a tumor

In yet another exemplary embodiment, the computer-implemented method further includes: determining a location of each of first or second gamma rays incident on a two-dimensional array of absorbing crystals, and determining an energy of each of the first or second gamma rays incident on the crystals.

In yet another exemplary embodiment, the computer-implemented method further includes: storing the location of each of the first or second gamma rays incident on the crystals, assigning a grey scale to each of the first or second gamma rays based on the energy determined, and associating the assigned grey scale with the first two-dimensional image or the second two-dimensional image.

In yet another exemplary embodiment of the computer-implemented method, the tumor includes a radiopharmaceutical.

In yet another exemplary embodiment, the needle includes a gamma-emitting source.

In yet another exemplary embodiment, the radiopharmaceutical is associated with a first isotope.

In yet another exemplary embodiment, the gamma-emitting source is associated with a second isotope.

In yet another exemplary embodiment, the first isotope comprises Tc-99m.

In yet another exemplary embodiment, the second isotope comprises Ce-139.

In yet another exemplary embodiment, the first isotope emits the first gamma rays.

In yet another exemplary embodiment, the second isotope emits the second gamma rays.

In yet another exemplary embodiment, the first gamma rays have approximately 140 keV in energy.

In yet another exemplary embodiment, the second gamma rays have approximately 166 keV in energy.

In yet another exemplary embodiment, the displaying step includes generating a third two-dimensional image using the first two-dimensional image and the second two-dimensional image.

In yet another exemplary embodiment, the step of generating a third two-dimensional image includes overlapping the first image on the second image or overlapping the second image on the first image.

In another embodiment, a system includes one or more processors and memory, where the one or more processors fetch instructions from the memory. The instructions cause the one or more processors to: (a) generate a first two-dimensional image at a first energy level, (b) generate a second two-dimensional image at a second energy level, (c) display simultaneously the first and second two-dimensional images, and (d) select display coefficients for the first and second two-dimensional images.

In another embodiment, a non-transitory computer-readable storage medium stores one or more programs configured for execution by a computer. The one or more programs include instructions to: (a) generate a first two-dimensional image at a first energy level, (b) generate a second two-dimensional image at a second energy level, (c) display simultaneously the first and second two-dimensional images, and (d) select display coefficients for the first and second two-dimensional images.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of the specification, illustrate embodiments of the invention and together with the description serve to explain the principles of the invention. In the drawings:

FIG. 1 is a conceptual front view of a preferred embodiment of a gamma-guided stereotactic localization system according to the present invention including a gamma camera crystal, a stereo imaging system including a set of oppositely viewing slant-hole collimators, and a grid localization system with a fiducial marker source.

FIG. 2 is a top view of the grid localization system and fiducial marker source which form a portion of the gamma-guided stereotactic localization system of FIG. 1.

FIG. 3 is a side view of an inner tube that contains a gamma emitting marker source.

FIG. 4 is an end view of the inner tube depicted in FIG. 3.

FIG. 5 is a side view of an outer cannula that will contain the inner tube of FIG. 3 and which will be in contact with the object to be imaged.

FIG. 6 is an end view of the outer cannula depicted in FIG. 5.

FIG. 7 is a phantom side view of a biopsy needle guide including an inner tube with a gamma emitting marker source and enclosed in an outer cannula.

FIG. 8 is an end view of the biopsy needle guide of FIG. 7.

FIG. 9 is a conceptual front view of a second embodiment of a gamma-guided stereotactic localization system according to the present invention including a gamma camera crystal, a stereo imaging system including a parallel hole collimator and a set of oppositely viewing slant-hole collimators, and a grid localization system with a fiducial marker source.

FIG. 10 is a graph depicting gamma camera images of point sources at various heights when imaged with a slant-hole collimator pair aligned such that the seam where the two collimators are joined was placed directly under the point sources.

FIG. 11 is a graph depicting two images of the point sources located at various heights which was used to determine the separations of the point sources.

FIG. 12 is a graph of the separation of the two images of the source versus the actual height.

FIG. 13 illustrates an exemplary system block diagram of an imaging system 1300 executing a marker processing and identification application in accordance with some embodiments.

FIG. 14 illustrates an exemplary module block diagram in accordance with some embodiments.

FIG. 15 illustrates an exemplary process flow in accordance with some embodiments.

FIGS. 16A-B illustrate an exemplary images generated using image display techniques of some embodiments.

FIG. 16C illustrates an exemplary image generated using an overlay images technique of some embodiments.

### DETAILED DESCRIPTION

Reference will now be made in detail to embodiments, examples of which are illustrated in the accompanying drawings. In the following detailed description, numerous non-limiting specific details are set forth in order to assist in understanding the subject matter presented herein. It will be apparent, however, to one of ordinary skill in the art that various alternatives may be used without departing from the scope of the present invention and the subject matter may be practiced without these specific details. For example, it will be apparent to one of ordinary skill in the art that the subject matter presented herein can be implemented on any type of standalone system or client-server compatible system containing any type of client, network, server, and database elements.

In some embodiments, provided is a gamma-guided stereotactic localization system for accurately locating and guiding biopsy equipment to cancerous lesions. The gamma-guided stereotactic localization system is a functional or molecular breast imaging procedure that captures the metabolic activity of breast lesions through radiotracer uptake. A small amount of tracing agent is delivered to a patient, and in turn is absorbed by all cells in the body. The tracing agent emits invisible gamma rays, which are detected by a gamma camera and translated into a digital image of the breast. Due to the higher metabolic activity of cancerous cells, these cells absorb a greater amount of the tracing agent and are revealed as “hot spots.” This molecular breast imaging technique can help doctors more reliably differentiate cancerous from non-cancerous cells. While other adjunct modalities, such as MRI and ultrasound, image the physical structure of the breast, the gamma-guided stereotactic system of the present invention captures the cellular functioning of the breast tissue.

Stereotactic localization uses at least two images of an object taken at different angles to determine the three dimensional location of a region-of-interest in that object, relative to the imaging system coordinates. It is desirable to have a gamma-guided localization system for use in the gamma imaging of suspected cancer to guide a physician in the removal of tissue samples for biopsy.

It is also desirable to correlate the location of the region relative to the camera with the location of the region in the object and to provide a positioning system that can be used to support and accurately position other hardware in the vicinity of the object. Once the location is correlated with the object, it can be used, for example, for positioning a needle in a suspected tumor to collect a tissue sample for biopsy.

In an effort to reduce that chance for error in this localization system, verifying that the calculated location does indeed correspond to the location of the lesion is also desirable. This may be accomplished using a marker to be placed in the object and the imaging system used to image this marker at that location. That image can then be compared with the image of the region of interest. In order to be imaged by the gamma camera, these markers are radioactive so that the marker can be seen in the image of the object.

In order to better identify the marker and ensure the accuracy of the region identified for biopsy, or some other interventional procedure, the imaging system may include a feature for overlaying two simultaneous co-registered images using different color palettes, with real-time contrast adjustment. Co-registering the images occurs by capturing them simultaneously using the same gamma ray optics (i.e., same collimator and gamma detector). By overlaying co-registered images with different color palettes, e.g., each color palette potentially corresponding to a tumor and a needle to be used in the biopsy/interventional procedure, and displaying the images on a screen that medical personnel view in order to determine the location of the tumor with respect to the needle, a more accurate determination of the location of the tumor in relation to the location of the needle may be made. Because cancerous cells at the location of the tumor are characterized by enhanced metabolic activity, a radiopharmaceutical injected into a patient's body is concentrated in greater quantity at the site of the tumor. Under gamma-guided imaging, the injected radiopharmaceutical is capable of detection because of the gamma rays it emits. A different type of gamma-emitting source may be used for the needle, i.e., a gamma-emitting source that emits gamma rays at a different energy than the radiopharmaceutical. Using gamma-guided imaging, which detects emitted gamma rays and displays them on a display screen, the injected radiopharmaceutical may be distinguished visually from the needle when, for example, a different color palette is used to identify the radiopharmaceutical than is used to identify the gamma-emitting source in the needle. Achieving more accurate spatial correlation is at least one advantage of implementing this feature in the imaging system.

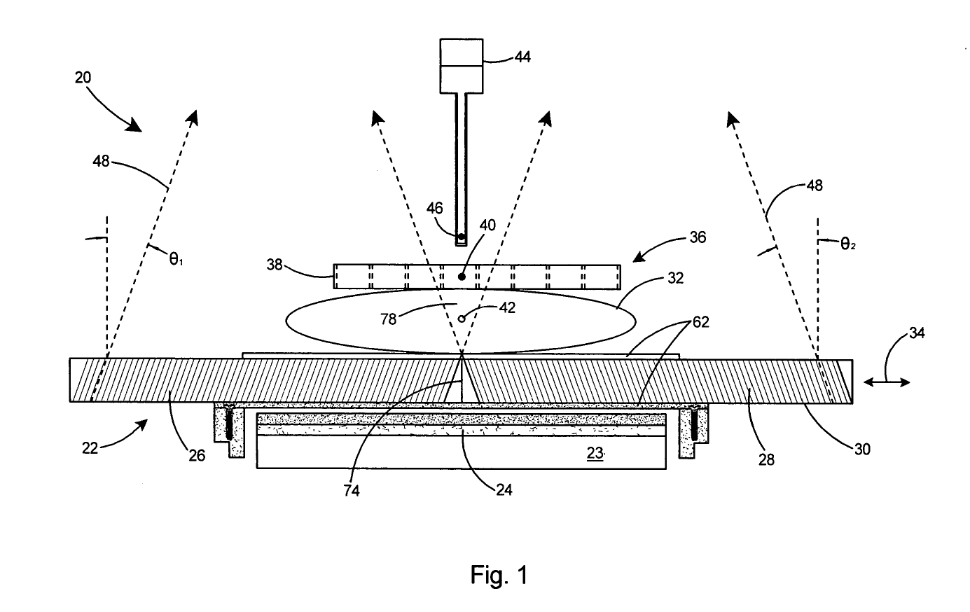

FIG. 1 illustrates an exemplary embodiment of a gamma-guided stereotactic localization system 20. The gamma-guided stereotactic localization system 20 includes a stereo imaging system 22 composed of a gamma camera 23 including a gamma camera crystal 24 and a set of oppositely viewing slant-hole collimators. A first or left side slant-hole collimator 26 and a second or right side slant-hole collimator 28 are coplanar with each other and joined at their ends form a side-by-side collimator set 30 that is movable with respect to the gamma camera crystal 24 and the body part or object 32 to be imaged. The collimator set 30 is movable left to right in FIG. 1 as shown by directional arrow 34. A positioning system 36 includes a grid localization system or plate 38 that is rigidly mounted above the object 32 to be imaged. A radioactive fiducial source 40 that can be imaged by the stereo imaging system 22 is accurately mounted in the positioning system 36. The stereo imaging system 22 is used to accurately identify a region-of-interest 42, such as a suspected cancerous lesion, in the object 32 to be imaged. The positioning system 36 is placed adjacent to the object 32 to be imaged and is used to support and accurately position other hardware, such as the biopsy needle guide 44, in close proximity to the object 32. A marker source 46, shown in the end of the biopsy needle guide 44, can be inserted into the object 32 at the calculated location of the region of interest 42 or lesion and used to verify that the calculated location is the actual location of the lesion. As shown by the angles of the conceptual camera viewing lines 48 in FIG. 1, the stereotactic viewing angles Θ₁ and Θ₂ are at +/−20 degrees with respect to a line perpendicular to the face of the gamma camera crystal 24. Left side slant-hole collimator 26 therefore views at a 20 degree angle to the right and right side slant-hole collimator 28 views at a 20 degree angle to the left.

The stereotactic gamma-guided localization method of some embodiments involves three steps, including localization, correlation, and verification. As shown in FIG. 1, the localization system includes a gamma camera having a gamma crystal 24 with a set of slant-hole collimators 26 and 28 that serve as the stereo viewing system. The positioning system 36 includes a grid localization system 38 with a fiducial source 40 therein and is placed adjacent to the object 32 to be imaged and held rigidly in place. The verification system includes a gamma emitting marker 46 that can then be placed by a biopsy needle guide 44 or similar tool at the calculated location of the region of interest 42 and imaged by the stereo imaging system 22 to verify that the inserted marker 46 coincides with the region of interest 42.

The location of the fiducial source 40 relative to the stereo imaging system 22 is calculated from the gamma camera images. The location of the region-of-interest 42 relative to the stereo imaging system 22 is then calculated to locate the region-of-interest 42 relative to the fiducial source 40. The positioning system 36 can then be used to accurately position and support any other hardware, such as the biopsy needle guide 44 shown in FIG. 1, which may be positioned at the region of interest 42, by measurements from the fiducial source 40. The marker source 46 is then to be used to verify that the calculated location of the region of interest 42 corresponds to the actual location.

The stereotactic gamma-guided localization method of some embodiments involves three steps including localization, correlation, and verification. More specifically, the localization step includes 1) placing a positioning system adjacent to the object to be imaged, 2) taking a pair of stereo images of the object; 3) determining the region of interest in each of the stereo images, and 4) calculating the three dimensional (X, Y, and Z) location of the region of interest. Correlation includes 1) locating the fiducial marker in each of the images, 2) calculating the (X, Y and Z) location of the fiducial marker, 3) locating the region-of-interest relative to the fiducial marker within the positioning system, and 4) utilizing the positioning system to locate and support any other hardware that may be positioned at the region of interest. Verification includes 1) positioning a marker source at the calculated location of the region of interest, and 2) imaging with the stereo imaging system to verify that the calculated location indeed corresponds to the actual location.

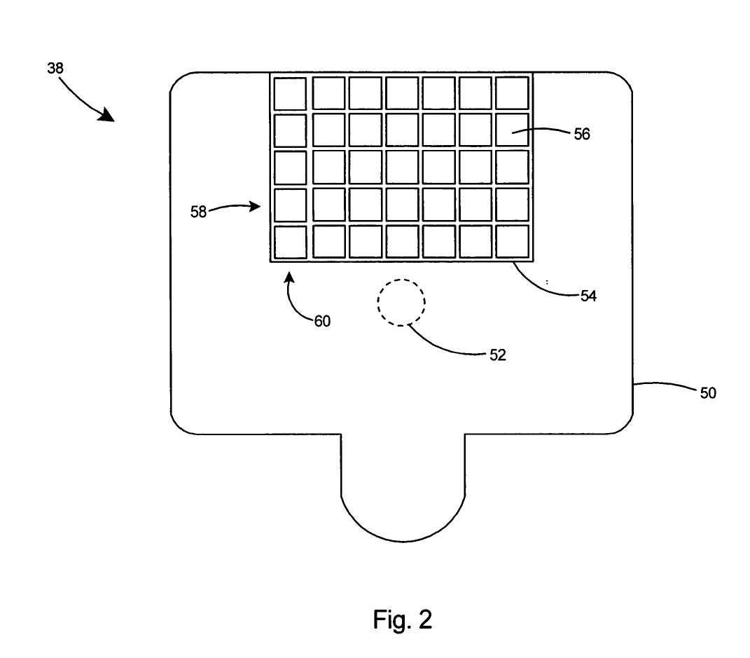

FIG. 2 illustrates an exemplary top view of a grid localization system 38 that forms a portion of the gamma-guided stereotactic localization system of the present invention. The grid localization system 38 includes a grid support or shield 50, a cavity 52 for accepting a fiducial source 40 therein, and a grid 54 with a plurality of grid openings 56 therein arranged in rows 58 and columns 60. The grid localization system 38 enables a qualified physician to perform a gamma-guided breast biopsy using a standard breast biopsy needle kit. The grid shield 50 is typically used to immobilize the breast during an imaging procedure.

The grid localization system 38 is used to locate the area of the breast or other body part that is directly above the suspected lesion. The grid 54 will be correlated with the location of the lesion that has been determined during the localization procedure via the fiducial marker 40. The fiducial marker 40 is imaged at the same time as the lesion and the location of the lesion relative to the fiducial marker 40 is calculated. The grid localization system 38 will also serve to stabilize the biopsy needle system during the gamma-guided breast biopsy procedure.

The fiducial marker 40 is typically inserted into a cavity 52 in the grid support 50. The fiducial marker 40 is mechanically registered to the grid system and is used to correlate the location of the suspected lesion as determined by the stereo imaging system 22 and the grid localization system 38. This allows the physician to determine the location of the suspected lesion by measurements from the grid localization system 38. Preferably, the fiducial marker 40 is a radioactive source of Ce-139 inserted into the cavity 52 on the grid support 50. Preferably, the activity level of the fiducial marker 40 is sufficient to be seen simultaneously with the lesion in the imaged object 32 but low enough as to present no significant risk to the patient. The fiducial marker is typically refreshed annually or as indicated by an expiration date on the package.

The gamma-guided stereotactic localization system 20 includes a cassette 62 (see FIG. 1) in which the collimator set 30 slides, and a computer including software having a biopsy protocol that is used to determine the three dimensional location of the lesion from the gamma camera images. The biopsy protocol takes the inputs from the stereo imaging system 22 and determines the three dimensional location of the lesion. Referring to FIG. 2, the biopsy protocol is also used to correlate the location of the lesion to the grid system 38 such that the biopsy needle 44 can be positioned at the appropriate location within the grid 54 and inserted into the object 32. The biopsy protocol will also allow images to be taken during the biopsy procedure, to determine that the biopsy needle 44 has been positioned correctly.

The gamma-guided stereotactic localization system 20 includes a marker validation procedure whereby the gamma emitting marker/obturator 46 (see FIG. 1) is used to determine that the incision in the object 32 has been made to the correct location. The marker 46, which will contain a radioactive source (e.g., Ce-139), will be inserted into the object 32 after the incision is made and the object 32 imaged with the gamma camera. The sliding collimator set 30 is positioned such that the gamma emitting marker 46 can be imaged through both halves 26 and 28 of the slant-hole collimator. The two images of the gamma emitting marker 46 will appear in the location of the lesion if the incision has been made to the correct location, confirming that the biopsy is taken at the site of the suspected lesion 42.

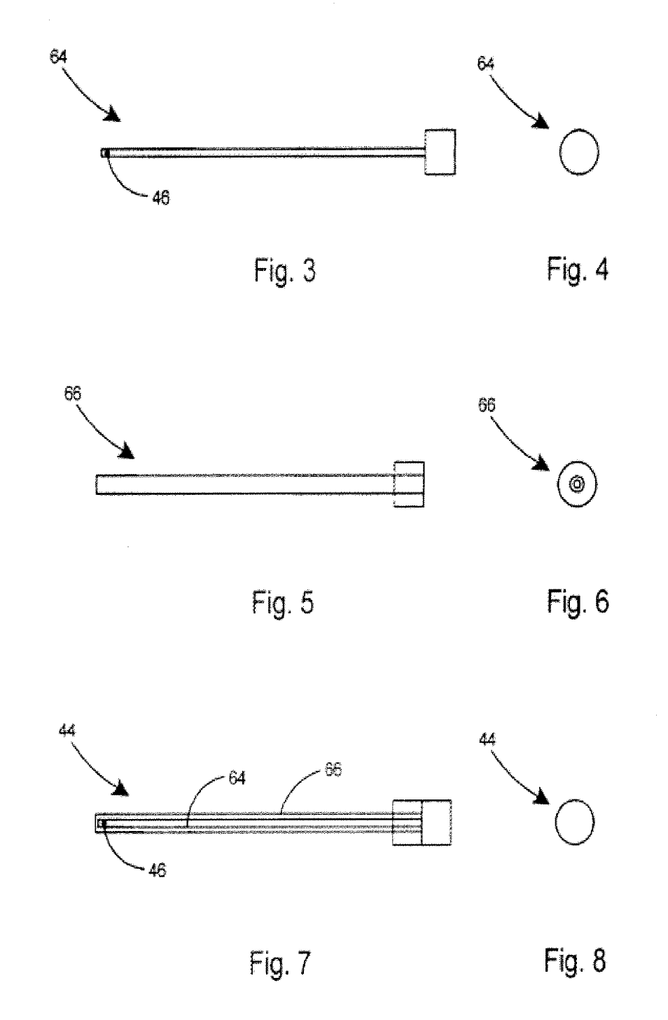

FIGS. 3-8 illustrate an exemplary gamma emitting marker 46 as part of a source holder/biopsy needle guide 44 with at least two parts, including an inner tube 64 that contains the gamma emitting marker 46 and an outer cannula 66 that will be in contact with body fluids in the object 32 to be imaged. The inner tube 64, as shown in FIGS. 3 and 4, is fitted into the outer cannula 66, depicted in FIGS. 5 and 6, to form the source holder/biopsy needle guide 44 with gamma emitting marker 46 as shown in FIGS. 7 and 8. The source holder 44 preferably contains a long half-life isotope such that it can be used for an extended period, and therefore used for a number of imaging and biopsy procedures. The half-life of the isotope is preferably of the order of a few months to a year. Preferably, the activity of the gamma emitting marker 46 is such that it can be viewed conveniently in the image without causing significant radiation exposure, or overwhelming the image of the imaged object 32. This activity will depend on the gamma ray attenuation of the material used for the biopsy needle guide/source holder 44 and the outer sleeve 66. Preferably the expected activities of the gamma emitting source 46 may be in the range of 5-10 microcurie. For reference, the typical specific activity of the lesion may be 2 microcurie/cc and that of the imaged object may be 0.3 microcurie/cc.

The energy of the gamma rays emitted from the gamma emitting source 46 may be equal to or higher than the isotope used for the imaging of the object 32. The isotope in the radiopharmaceutical within the object may be, for example, Tc-99m, which has a gamma-ray energy of 140 keV. Examples of isotopes that may be used as the gamma emitting source 46 and have gamma ray emissions with energy greater than 140 keV, as well as a relatively long half-life, are Ce-139 with an energy of 166 keV and a half-life of 137.6 days, and Te-123m with an energy of 159 keV and a half-life of 120 days. These isotopes can be produced as solid materials and encapsulated into the biopsy needle guide/source holder 44.

An alternative embodiment for the gamma emitting source 46 is a one piece design (not shown) that can be filled with a radioactive liquid. The radioactive liquid will have a short half-life and can therefore be safely discarded after an appropriate time, typically 10 half-lives. This marker will be disposed of after each use as biological hazardous material.

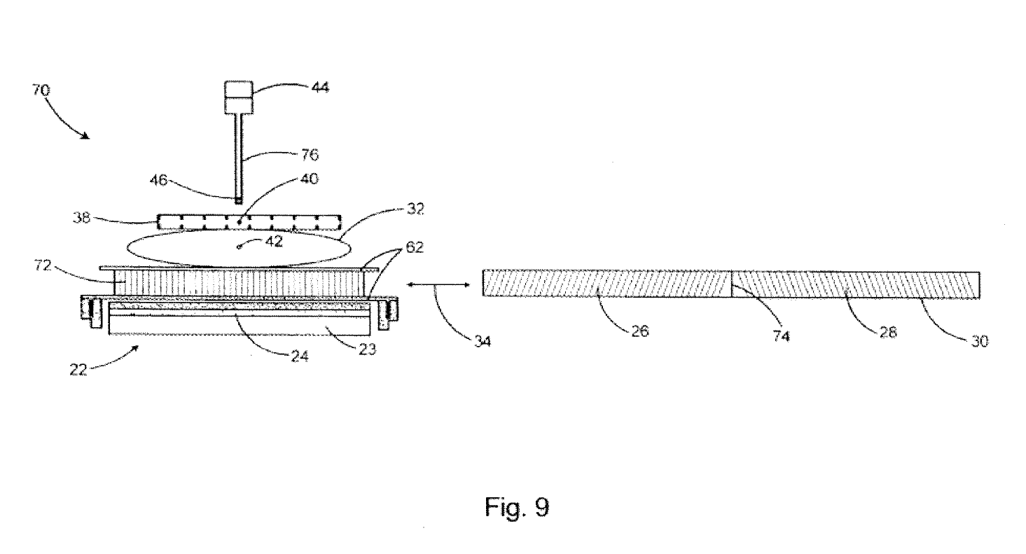

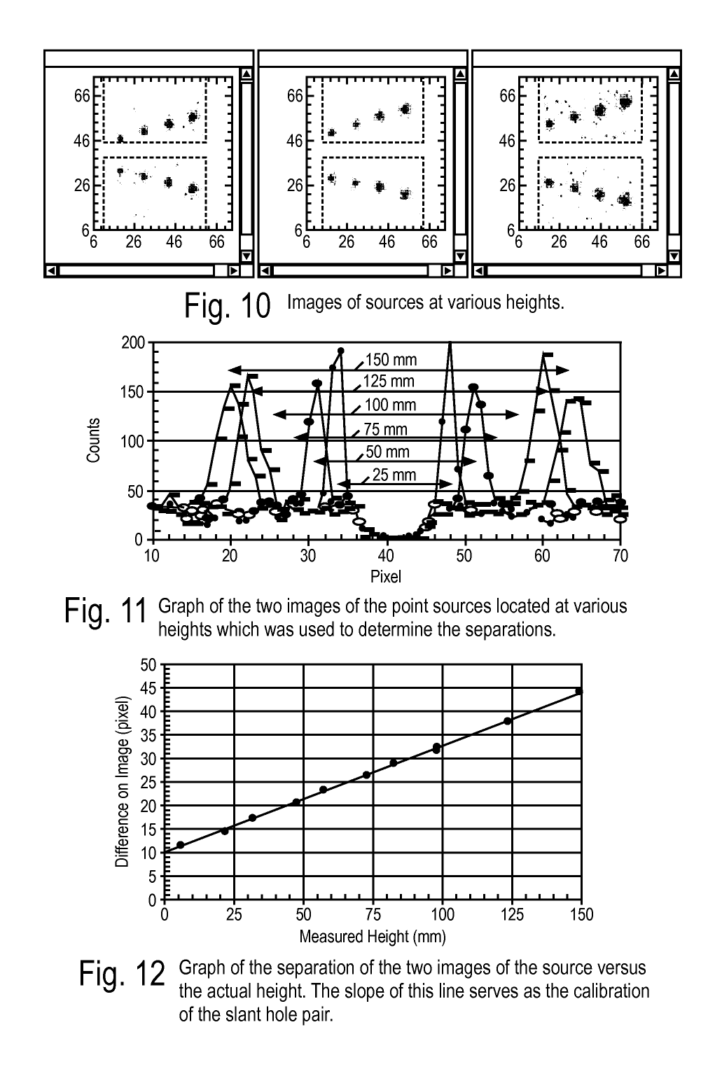

FIG. 9 illustrates an alternative embodiment of a gamma-guided stereotactic localization system 70 that includes a parallel-hole collimator 72 and a stereo-collimator assembly 30 made from a pair of 20-degree slant-hole collimators 26 and 28. Both the parallel-hole collimator 72 and the slant-hole stereo-collimator assembly 30 slide in a cassette 62 that mounts on the top of the gamma camera 24. The parallel-hole collimator 72 is slid into the cassette 62 and used to produce a “scout” image. This image is used to determine the X and Y location of the region-of-interest 42. The slant-hole collimator assembly 30 then slides into the cassette 62. Images are then taken to determine the Z location (depth) of the suspected regions.

A region of interest can be identified in the gamma-guided stereotactic localization system 70 of FIG. 9 by either of two methods. In one method, the location of the region-of-interest 42 in the images taken with the parallel-hole collimator 72 and the slant-hole collimators 30 are used to calculate the location of the suspected region. The X and Z location of the region can be determined from the following expressions:

$$\begin{matrix}X = \frac{{X}^{L} + {X}^{R}}{2} & ( 1 ) \\ Z = \frac{( {X}^{L} - {X}^{R} )}{2 tan θ} & ( 2 )\end{matrix}$$

where X^{L} and X^{R} are the X locations of the region-of-interest in the left and right viewing images and e is the slant-hole angle, which in an embodiment is 20 degrees. In a second method, the location of the region-of-interest 42 is determined interactively by aligning a source, such as fiducial source 40 in FIG. 9, with the location of the region, as determined by the images from the parallel-hole collimator 72 and slant-hole collimators 30. For small isolated regions of uptake this method is preferred. For large regions or multi-focal uptake, the first method is preferred.

In order to calculate the location of the region of interest, the slant-hole collimator assembly 30 is slid into the cassette. Two images are then taken to determine the depth or Z location of the suspected region or regions.

When determining the location of a region-of-interest interactively by the second method, the parallel-hole collimator 72 is used and a source 46 is positioned above the object 32 and aligned with the region to determine the location in the X and Y dimensions. This can either be a sealed source such as source 46 depicted in FIG. 9 or a drop of Tc-99m that is moved above the object 32. To determine the Z location, the slant-hole collimator assembly 30 is positioned such that the line 74, where the slant-hole sections 26 and 28 are joined, is placed under the region-of-interest 42. A source 46 is placed in a vessel that can be inserted into the breast, such as the biopsy needle portion 76 of biopsy needle guide 44, and the image of the source 46 is monitored in both stereo views. The correct Z location is determined by aligning the image of the source 46 and the image of the region-of-interest 42 in the two views.

The relationship between the height of the point source and the separation of the images of the point source created by the two halves of the slant-hole collimator pair 30 could be determined from the geometry of the stereo imaging system. The relationship between the height above the collimator assembly 30 and the separation of the images produced by the two slant-hole collimators 26 and 28 is given by:

h=s/2 tan θ-h₀=(s-s₀)/2 tan θ (3)

where h is the height above the collimator assembly (in mm), s is the separation of the two images (in mm), θ is the slant-hole angle, h₀ (=s₀/2 tan θ) is the height of the collimator above the gamma camera crystal 24 and so is the separation of the two images when the source is on the surface of the collimator. The separation of the images can be determined in terms of the pixel separation, by the equation:

s=p·pixel spacing (4)

where p is the separation in pixels and the pixel spacing for a preferred embodiment of the gamma camera is 3.2 mm/pixel. The height above the gamma camera crystal 24 can be determined from the design of the camera system. It is the sum of the size of the collimator (siz), the separation between the collimator and the camera (sep) and the depth of the crystals within the camera (dep). The total height can be determined from:

h₀=siz+sep+dep=27. 2+5.1+6.2=38.5 (5)

where all dimensions are in mm. Using these values, an expression for the height of the source can be determined from the separation of the images of the point sources from the following equation:

h=p·(pixel spacing/2 tan 20°)−(siz+sep+dep)

Substituting the values of these parameters gives an equation of the form:

h=p·4.4-38.5 (6)

To determine if the relationship between the height of the point source and the separation of the images of the source can be reliably determined from the relationship given in equation (6) above, measurements were made of the separation of the images of point sources are various heights and compared with the value determined from the equation. Four sources were placed on spacers of various heights and placed on the detector. Images were taken of these four sources with the slant-hole collimator pair 30 aligned such that the seam 74 where the two collimators 26 and 28 are joined was placed directly under the point sources. This was repeated three times with different sets of spacers each time. The images are shown in FIG. 10.

The images indicate that the separation of the images produced by the two halves 26 and 28 of the slant-hole collimator pair 30 scales directly with the height of the source above the collimator assembly 30. The separation of the point sources was determined for these three images. Each pair of images was projected onto the axis parallel to their separation and the peak locations were determined. Examples of these projections are given in FIG. 11. A center of gravity calculation was used to determine the peak location for each of the pairs, such that sub-pixel resolution could be achieved. The separation in these peaks determined from the center of gravity calculation was plotted versus the actual height of the source above the collimator assembly 30 in FIG. 12 to determine the relationship between the height and the separation on the image. Also plotted in FIG. 12 is equation (5), the equation used to predict the relation between the height and separation. The results plotted in FIG. 12 show that the measured relationship between the height and the separation compares well with the expected values.

Measurements were made to determine the field-of-view (FOV) that could be viewed by the two slant-hole collimators 26 and 28 simultaneously (see FIG. 1). These measurements were compared with geometric considerations to determine if the field of view could be reliably predicted. The field of view seen by both of the slant-hole collimators is determined by the slant-hole angle. The width of this field of view is given by the following expression:

FOV=2·h·tan θ (7)

where 2*tan e˜0.73. If the separation of the sources is to be determined and a center of gravity calculation used to determine the pixel location of the sources, the blurring of the collimator should be taken into account. The blurring of the collimator with height can be approximated by a linear function and added to the expression to give a better approximation of the effective field of view of the overlap region. Assuming the width of a point source (w) increases linearly with height, the field of view would be decreased by this width. The expression for the field of view would then be expressed by:

FOV=2·h·tan θ-k·h (8)

where w=k*h and k is the coefficient that describes the increase in width with height. The value of k for the preferred embodiments of the collimators 26 and 28 described herein is approximately, k˜0.24. This reduces the size of the field of view by approximately 30%.

Tests with point sources and phantoms were conducted to determine the accuracy and functionality of the sliding slant-hole collimator system. As described herein, point sources were used to determine the spatial resolution and sensitivity of the collimator system, the field-of-view of the slant-hole pair, and to demonstrate an ambiguity that exists with stereotactic imaging. Gelatin breast phantoms were used to investigate the accuracy with which a region-of-interest could be located and removed from breast tissue using the collimator system for guidance. The results indicate the successful simulated biopsies could be performed with the gamma-guided stereotactic localization systems 20 and 70 of the described embodiments.

The spatial resolution of the gamma-guided stereotactic localization systems 20 and 70 of the described embodiments has a large impact on the accuracy of the determination of the Z-coordinate or depth of the region-of-interest because of the angle involved in the stereo view. As can be seen from equation (2), the spatial resolution in the Z dimension is a factor of ½tan θ=1.4 times the resolution in the X dimension.

As described herein, a gamma-guided stereotactic localization system consisting of a sliding slant-hole collimator arrangement can be used to produce stereo images from a compact gamma camera and to determine the three dimensional location of a region of interest. The gamma-guided stereotactic localization system of the described embodiments has been successfully used to perform vacuum assisted biopsy procedures on gelatin breast phantoms.

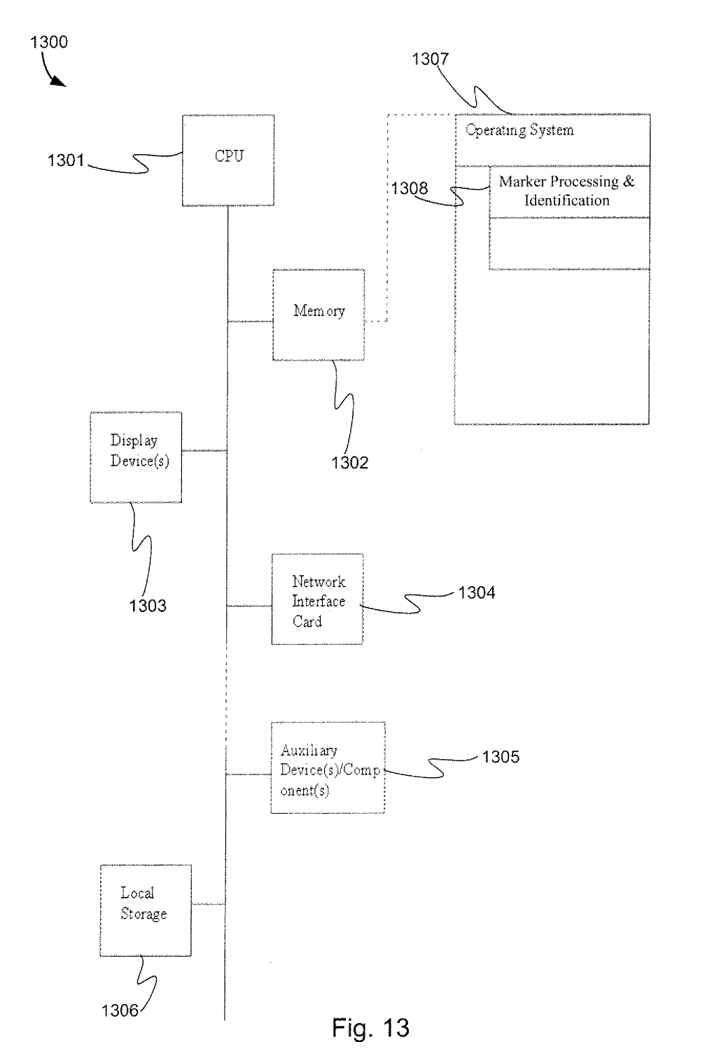

FIG. 13 illustrates an exemplary system block diagram of an imaging system 1300 executing a marker processing and identification application 1308 in accordance with some embodiments. The imaging system 1300 may include a computer processing unit (CPU) 1301, memory 1302 (e.g., volatile or non-volatile), display device(s) 1303, network interface card (NIC) 1304, an interface for auxiliary device(s)/component(s) 1305, and local storage 1306 (e.g., non-volatile). Each of the foregoing may communicate using a shared bus architecture. An operating system 1307 may reside in local storage 1306, remotely on a network accessible by the NIC 1304, and/or memory 1302. Instructions being executed by the CPU 1301 may be fetched from memory 1302 and may include instructions from one or more modules of the marker processing and identification 1308 application and/or one or more other applications. The system 1300 may be contained within the stereo imaging system 22, a portable device, laptop computer, desktop computer, server, or some other system capable of housing the components 1301-1306.

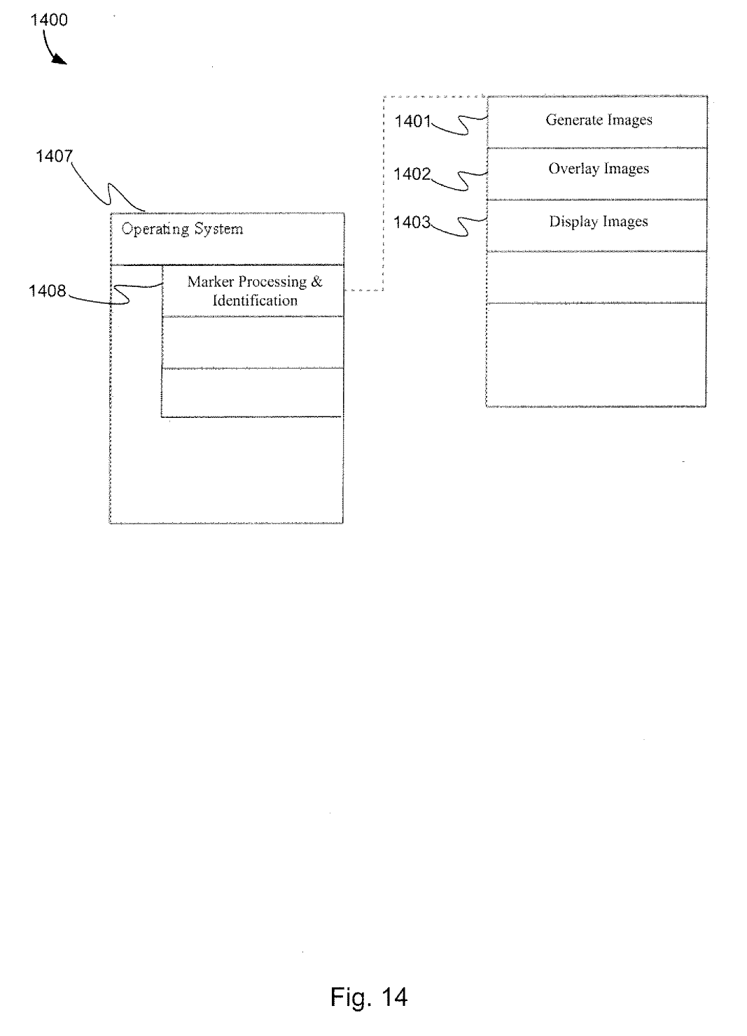

FIG. 14 illustrates an exemplary module block diagram 1400 in accordance with some embodiments. The module block diagram 1400 includes a marker processing and identification application 1408 that may include a generate images module 1401, an overlay images module 1402, and a display images module 1403. The marker processing and identification application 1408 may include other modules for carrying out one, of its purposes. Namely, generating gamma ray images based on detected gamma rays that have different energies, processing the generated images so that upon display the different energies are represented visually using different color palettes, and overlaying the processed images for visual display so that the location of one or more markers could be determined accurately relative to the location of one or more lesions.

In some embodiments, the generate images module 1401 may receive image data over, for example, a system bus via local storage 1306 and/or memory 1302. The image data may correspond to one or more images captured by the stereo imaging system 22 and, more specifically, by the gamma camera 23. Because gamma ray images may be produced by determining the number of gamma rays that are incident on a two-dimensional array (e.g., N[(x, y)]) of gamma ray absorbing crystals, the image data that represents a gamma ray image may be represented by a two-dimensional array of numbers. The numbers in the array may correspond to the number of gamma rays incident on the gamma ray crystal 24 at an (x, y) location. The energy of the gamma rays may also be determined by, for example, the stereo imaging system 22 at the time of detection of the incident gamma rays. Separate image data may be stored for the incident gamma rays in various energy bands (e.g., [N₁(x,y), N₂(x,y)]).

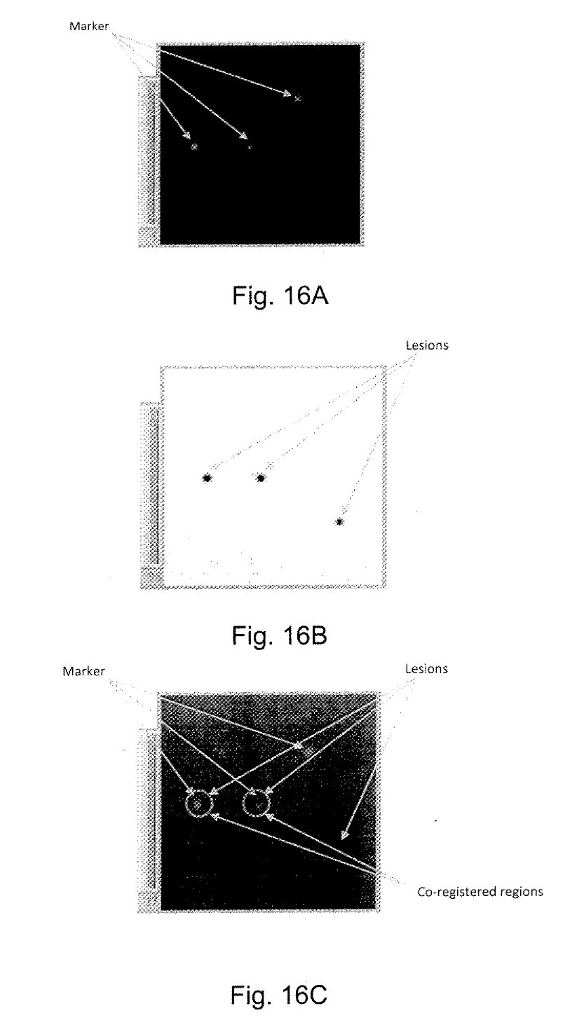

In some embodiments, the display images module 1403 may access the image data stored in local storage 1306 and/or memory 1302 for the purpose of displaying the image corresponding to the image data. When displaying the image, the display images module 1403 may assign a particular pixel color and/or intensity to one or more particular locations of a display screen/device 1303. The pixel color and/or intensity may be based on the data in the two-dimensional array that is related to the number and/or energy of gamma rays detected in a particular (x,y) location. For example, a lack of any gamma rays (i.e., g_{zero}) in an (x,y) location may be represented by the color black, whereas the maximum number of gamma rays (gₘₐₓ) detected in an (x,y) location may be represented by the color white. Any gamma rays detected within that range may be represented by a shade of gray relative to whether the number of gamma rays are closer to g_{zero} or gₘₐₓ. The number of available shades of gray may be represented by a variable K. An exemplary illustration of an image created using this technique is shown in FIG. 16A. The image shown in FIG. 16A illustrates a white on black background image, where the pixels shown in white/grey correspond to the number of and/or energy intensity of gamma rays of one or more markers.

In some embodiments, the image data corresponding to a number of gamma rays between 0 and (gₘₐₓ/K)-1 may be assigned to a black pixel color. Also, the image data corresponding to a number of gamma rays between gₘₐₓ/K and [2*(gₘₐₓ/K)]-1 may be assigned to the darkest shade of grey. Furthermore, the image data corresponding to a number of gamma rays between (K-2)*(gₘₐₓ/K) and [(K-1) * (gₘₐₓ/K)]-1 may be assigned to the lightest shade of grey. Finally, the image data corresponding to a number of gamma rays between (K-1) * (gₘₐₓ/K) and gₘₐₓ may be assigned to the color white.

In some embodiments, a lack of any gamma rays (i.e., g_{zero}) in an (x,y) location may be represented by the color white, whereas the maximum number of gamma rays (gₘₐₓ) detected in an (x,y) location may be represented by the color black. Any gamma rays detected within that range may be represented by a shade of gray relative to whether the number of gamma rays are closer to g_{zero} or gₘₐₓ. The number of available shades of gray may be represented by a variable K. An exemplary illustration of an image created using this technique is shown in FIG. 16B. The image shown in FIG. 16B illustrates a black on white background image, where the pixels shown in black/grey correspond to the number of and/or energy intensity of gamma rays of one or more lesions.

In some embodiments, the one or more markers and lesions may be rendered graphically in the display area using the OpenGL API. Shape/graphics object models of the markers and lesions may be constructed using the basic objects of OpenGL such as, for example, points, lines, polygons. Other features of the OpenGL API may also be invoked such as, for example, geometric primitives for describing objects mathematically, coding the color of shapes/graphics objects, arranging and modeling objects in a 3D space, shading a shape/graphics object smoothly, tracking the z-coordinates of shapes/graphics objects, operating on pixels, transforming shapes/graphics objects (e.g., rotating the shapes/graphics objects), and selecting a shape/graphics object and/or a specific portion of the display area.

In some embodiments, the overlay images module 1402 may process at least two images created by, for example, the display images module 1403 using the foregoing technique for displaying images as shown in FIGS. 16A-B. FIG. 16C illustrates an exemplary image processed by the overlay images module 1402. As shown in FIG. 16C the overlaid images may be used to illustrate the location of one or more markers with respect to one or more lesions. The overlaid images may be created using a combination of one image illustrating markers on a black background and another image illustrating lesions on a white background, for example. In some embodiments, the markers may be illustrated on a white background and the lesions on a black background. Other colors besides black and white, as well as shades of grey, may be used in other embodiments.

In some embodiments, a linear combination of the images may be used, such as that represented by equation (1) as:

I_{c}(x,y)=S/100x I₁(x,y)+(1-S/100)x I₂(x, y) (1)

where I_{c}(x,y) is the combined image; I₁(x,y) and I₂(x,y) are the original two images with. Energy 1 and 2, respectively; S is a number between 0 and 100 corresponding to a linear coefficient; and, (x,y) represents two-spatial dimensions. S may be entered/set manually by a user/operator of the gamma-guided stereotactic localization system 20 and, specifically, the stereo imaging system 22. FIG. 16A includes a graphical rendition of a slider scale where the value of S is 5. FIG. 16B includes a graphical rendition of a slider scale where the value of S is 95. FIG. 16C includes a graphical rendition of a slider scale where the value of S is 17. The slider scale may be adjusted by the user/operator manually using a graphical user interface, as shown in FIGS. 16A-C.

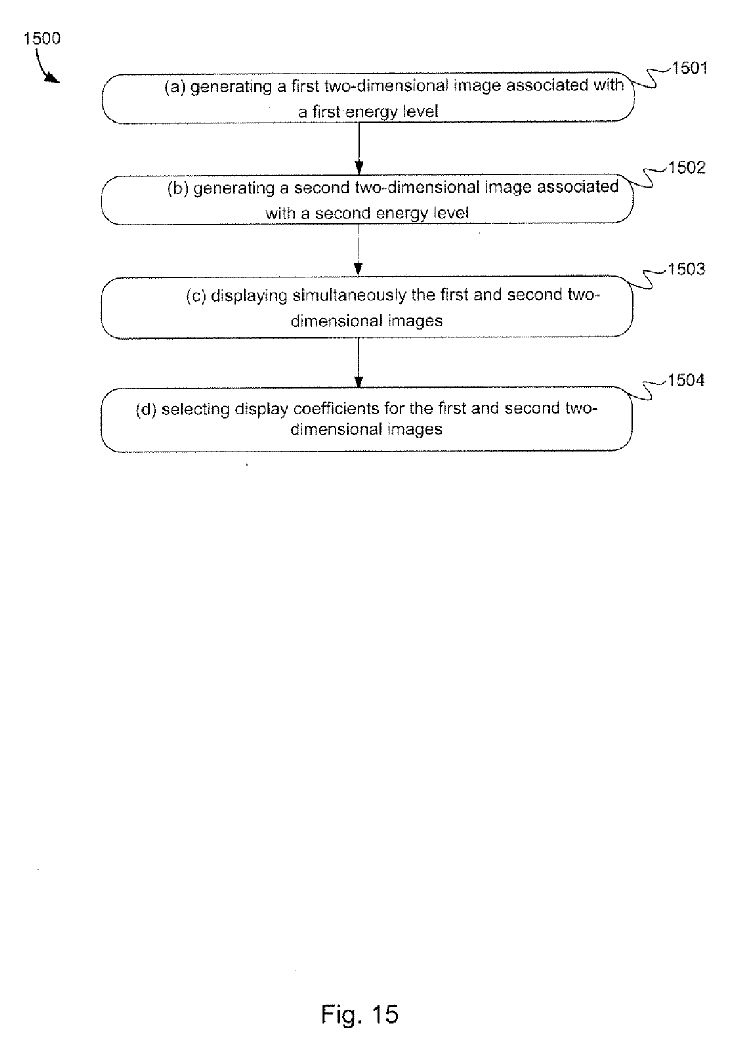

FIG. 15 illustrates an exemplary process flow 1500 in accordance with some embodiments. The exemplary process flow 1500 illustrates steps of a method that the marker processing and identification application 1408 may implement. The steps of the method include detecting a first two-dimensional image at a first energy level 1501, detecting a second two-dimensional image at a second energy level 1502, displaying simultaneously the first and second two-dimensional images 1503, and selecting display coefficients for the first and second two-dimensional images 1504. Other steps may be included between the foregoing steps 1501-1504. The steps 1501-1504 may be executed in different order and are not limited to the order in which they are recited.

It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents.

1. A computer-implemented method for co-registering gamma or positron images comprising:
(a) generating a first two-dimensional image associated with a first energy level;
(b) generating a second two-dimensional image associated with a second energy level;
(c) displaying simultaneously the first and second two-dimensional images; and
(d) selecting display coefficients for the first and second two-dimensional images.

2. The computer-implemented method of claim 1, wherein the first two-dimensional image corresponds to the location of a needle.

3. The computer-implemented method of claim 1, wherein the second two-dimensional image corresponds to the location of a tumor.

4. The computer-implemented method of claim 1, further comprising:
determining a location of each of first or second gamma rays incident on a two-dimensional array of absorbing crystals; and
determining an energy of each of the first or second gamma rays incident on the crystals.

5. The computer-implemented method of claim 4, further comprising:
storing the location of each of the first or second gamma rays incident on the crystals;
assigning a grey scale to each of the first or second gamma rays based on the energy determined; and
associating the assigned grey scale with the first two-dimensional image or the second two-dimensional image.

6. The computer-implemented method of claim 3, wherein the tumor includes a radiopharmaceutical.

7. The computer-implemented method of claim 2, wherein the needle includes a gamma-emitting source.

8. The computer-implemented method of claim 6, wherein the radiopharmaceutical is associated with a first isotope.

9. The computer-implemented method of claim 7, wherein the gamma-emitting source is associated with a second isotope.

10. The computer-implemented method of claim 8, wherein the first isotope comprises Tc-99m.

11. The computer-implemented method of claim 9, wherein the second isotope comprises Ce-139.

12. The computer-implemented method of claim 8, wherein the first isotope emits the first gamma rays.

13. The computer-implemented method of claim 9, wherein the second isotope emits the second gamma rays.

14. The computer-implemented method of claim 12, wherein the first gamma rays have approximately 140 keV in energy.

15. The computer-implemented method of claim 13, wherein the second gamma rays have approximately 166 keV in energy.

16. The computer-implemented method of claim 1, wherein the displaying comprises generating a third two-dimensional image using the first two-dimensional image and the second two-dimensional image.

17. The computer-implemented method of claim 16, wherein the generating a third two-dimensional image comprises overlapping the first image on the second image or overlapping the second image on the first image.

18. A system, comprising:
one or more processors;
memory;
the one or more processors fetching instructions from the memory, the instructions causing the one or more processors to:
(a) generate a first two-dimensional image associated with a first energy level;
(b) generate a second two-dimensional image associated with a second energy level;
(c) display simultaneously the first and second two-dimensional images; and
(d) select display coefficients for the first and second two-dimensional images.

19. A non-transitory computer-readable storage medium storing one or more programs configured for execution by a computer, the one or more programs comprising instructions to:
(a) generate a first two-dimensional image associated with a first energy level;
(b) generate a second two-dimensional image associated with a second energy level;
(c) display simultaneously the first and second two-dimensional images; and
(d) select display coefficients for the first and second two-dimensional images.
